(19) United States
(12) Patent Application Publication
(10) Pub. No.: US 2013/0142790 A1
(43) Pub. Date: Jun. 6, 2013

(54) Method of Treating Cancer Using a cMET and AXL Inhibitor and an ErbB Inhibitor

(71) Applicant: GlaxoSmithKline, King of Prussia, PA (US)

(72) Inventors: Tona M. Gilmer, Research Triangle Park, NC (US); James G. Greger, King of Prussia, PA (US); Li Liu, Collegeville, PA (US); Hong Shi, King of Prussia, PA (US)

(73) Assignee: GlaxoSmithKline, King of Prussia, PA (US)

(21) Appl. No.: 13/753,031

(22) Filed: Jan. 29, 2013

Related U.S. Application Data
(63) Continuation of application No. 12/435,473, filed on May 5, 2009.
(60) Provisional application No. 61/050,322, filed on May 5, 2008.

(51) Int. Cl. A61K 39/395; A61K 31/517; A61K 31/5377

(52) U.S. Cl. 424/133.1; 514/235.2

(57) ABSTRACT

The present invention relates to a method of treating cancer in a patient comprising administering to the patient therapeutically effective amounts of:
a) a compound of formula A:

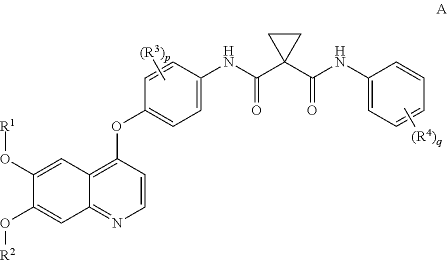

or a pharmaceutically acceptable salt thereof, wherein R¹—R⁴, p, and q are as defined; and
(b) an erbB inhibitor that inhibits erbB-1 or erbB-2 or erbB-3 receptor or a combination thereof. The method of the present invention addresses a need in the art with the discovery of a combination therapy that shows evidence of being a more effective therapy than previously disclosed therapies.

### RELATED APPLICATION DATA

This application is a continuation of U.S. Ser. No. 12/435,473, filed on May 5, 2009 which claims priority from U.S. Provisional Application No. 61/050,322, filed May 5, 2008.

### BACKGROUND OF THE INVENTION

The present invention relates to a method of treating cancer with an inhibitor targeting multikinases including cMET and AXL, in combination with an ErbB inhibitor.

Generally, cancer results from the deregulation of the normal processes that control cell division, differentiation, and apoptotic cell death. Apoptosis (programmed cell death) plays an essential role in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993), in particular, cellular signaling from the growth factor receptors of the erbB family.

ErbB-1 (also known as EGFR or HER1) and erbB-2 (also known as HER2) are protein tyrosine kinase transmembrane growth factor receptors of the erbB family. Protein tyrosine kinases catalyze the phosphorylation of specific tyrosyl residues in various proteins involved in the regulation of cell growth and differentiation (A. F. Wilks, Progress in Growth Factor Research, 1990, 2, 97-111; S. A. Courtneidge, Dev. Supp. 1, 1993, 57-64; J. A. Cooper, Semin. Cell Biol., 1994, 5(6), 377-387; R. F. Paulson, Semin. Immunol., 1995, 7(4), 267-277; A. C. Chan, Curr. Opin. Immunol., 1996, 8(3), 394-401).

ErbB-3 (also known as HER3) is a growth factor receptor of the erbB family that has a ligand binding domain but lacks intrinsic tyrosine kinase activity. HER3 is activated by one of its extracellular ligands (for example, heregulin (HRG)), then becomes a substrate for dimerization and subsequent phosphorylation by HER1, HER2, and HER4; it is this phosphorylated HER3 that leads to the activation of cell signaling pathways for mitogenic or transforming effects.

These receptor tyrosine kinases are widely expressed in epithelial, mesenchymal, and neuronal tissues where they play a role in regulating cell proliferation, survival, and differentiation (Sibilia and Wagner, Science, 269: 234 (1995); Threadgill et al., Science, 269: 230 (1995)). Increased expression of wild-type erbB-2 or erbB-1, or expression of constitutively activated receptor mutants, transforms cells in vitro (Di Fiore et al., 1987; DiMarco et al., Oncogene, 4: 831 (1989); Hudziak et al., Proc. Natl. Acad. Sci. USA., 84:7159 (1987); Qian et al., Oncogene, 10:211 (1995)). Increased expression of erbB-1 or erbB-2 has been correlated with a poorer clinical outcome in some breast cancers and a variety of other malignancies (Slamon et al., Science, 235: 177 (1987); Slamon et al., Science, 244:707 (1989); Bacus et al., Am. J. Clin. Path, 102:S13 (1994)). Overexpression of HRG and/or HER3 has been reported in numerous cancers, including gastric, ovarian, prostate, bladder, and breast tumors and is associated with poor prognosis (B. Tanner, J Clin Oncol. 2006, 24(26):4317-23; M. Hayashi, Clin. Cancer Res. 2008.14(23):7843-9.; H. Kaya, Eur J Gynaecol Oncol. 2008; 29(4):350-6;).

The modes of targeting erbB include the monoclonal anti-erbB-2 antibody trastuzumab, the anti-erbB-1 antibody cetuximab, the anti-erbB3 antibodies such as monoclonal antihuman erbB3 antibody mab3481 (commercially available from R&D Systems, Minneapolis, Minn.), and small molecule tyrosine kinase inhibitors (TKIs) such as the erbB-1/erbB-2 selective inhibitor lapatinib, and the erbB-1 selective inhibitors gefitinib and erlotinib. Nevertheless, these agents have shown limited activity as single agents (Moasser, British J. Cancer 97:453, 2007). It would therefore be an advantage in the field of oncology to discover treatments improve the efficiency of erbB inhibition for the treatment of a variety of cancers.

### SUMMARY OF THE INVENTION

In one aspect, the present invention is a method of treating cancer in a patient comprising administering to the patient therapeutically effective amounts of:

a) a compound of formula A:

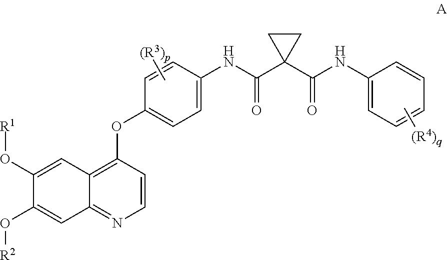

or a pharmaceutically acceptable salt thereof; and
(b) an erbB inhibitor that inhibits erbB-1 or erbB-2 or erb-3 receptor or a combination thereof; wherein,
R¹ is C₁-C₆-alkyl;
R² is C₁-C₆-alkyl or —(CH₂)ₙ—N(R⁵)₂;

### R³ is Cl or F;

### R⁴ is Cl or F;

each R⁵ is independently C₁-C₆-alkyl or, together with the nitrogen atom to which they are attached, form a morpholino, piperidinyl, or pyrazinyl group;
n is 2, 3, or 4;
p is 0 or 1; and
q is 0, 1, or 2.

The method of the present invention addresses a need in the art with the discovery of a combination therapy that shows evidence of being a more effective therapy than previously disclosed therapies.

### BRIEF DESCRIPTION OF DRAWINGS

FIG. 1 represents dose response curves of the cell growth inhibition by lapatinib and Compound I, alone, and in combination at 1:1 molar-to-molar ratio lapatinib:Compound I in OE-33 (cMET+ and HER2+) and NCI-H1573 (cMET+ and HER1+) cells in the presence of HGF.

FIG. 2 illustrates (left panel) the effects of HGF on the activity of lapatinib and the combination of lapatinib and Compound I at 1:1 molar-to-molar ratio lapatinib:Compound I in N87 HER2+ and cMET over-expressed tumor lines. FIG. 2 also illustrates (right panel) the inhibition of phosphorylation of cMET, HER2, HER3, AKT, and ERK by treatment of lapatinib and Compound I in the presence and absence of HGF as determined by western blot analyses.

FIG. 3 represents cell growth inhibition by lapatinib and Compound I, alone, and in combination at 1:1 molar-to-molar ratio lapatinib:Compound I in both BT474 (sensitive to lapatinib and trastuzumab) and BT474-J4 (resistant to lapatinib and trastuzumab) cells in the presence of HGF.

FIG. 4 illustrates apoptosis induction (DNA fragmentation and caspase 3/7 activation) by lapatinib and Compound I, alone, and in combination at 1:1 molar-to-molar ratio lapatinib:Compound I in both BT474 and BT474-J4 cells in the presence of HGF.

FIG. 5 represents cell growth inhibition and apoptosis induction by the combination of Compound I and lapatinib at different concentrations in BT474-J4 cells in the presence of HGF.

FIG. 6 illustrates 1) the inhibition of HER2 phosphorylation (pHER2) by lapatinib alone; 2) the inhibition of AXL phosphorylation (pAXL) by Compound I alone; and 3) the inhibition of pHER2 and pAXL as well as the diminution of: the phosphorylation of AKT (pAKT), the phosphorylation of ERK1/2 (pERK1/2), and cyclin D1 using the combination of Compound I and lapatinib in BT474-J4 cells.

FIG. 7 represents cell growth inhibition by trastuzumab and Compound I, alone, and in combination at 1:15 molar-to-molar ratio trastuzamab:Compound I after 5 days of compound treatment in both BT474 and BT474-J4 cells in the presence of HGF.

FIG. 8 represents dose response curves of the cell growth inhibition by erlotinib and Compound I alone, and in combination at 1:1 molar-to-molar ratio erlotinib:Compound I in NCI-H1648 (cMET+) and NCI-H1573 (cMET+ and HER1+) lung tumor cells in the presence of HGF.

FIG. 9 illustrates (left panel, labeled Cell Growth Inhibition) dose response curves of the cell growth inhibition by lapatinib and Compound I alone, and in combination at 1:1 molar-to-molar ratio lapatinib:Compound I in MKN45 (cMET+ and HER3-overexpression) tumor cells in the absence and presence of HRG. FIG. 9 also illustrates (right panel, labeled Western Blot Analysis) the inhibition of phosphorylation of cMET, HER1, HER3, AKT, and ERK by treatment of lapatinib and Compound I in the presence and absence of HRG as determined by western blot analyses.

### DETAILED DESCRIPTION OF THE INVENTION

In one aspect, the present invention relates to treating cancer using effective amounts of the compound of formula A and an erbB inhibitor wherein the compound of formula A is represented by the following formula:

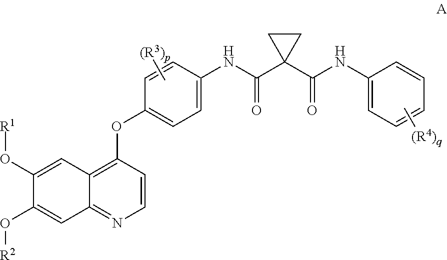

or a pharmaceutically acceptable salt thereof; wherein
R¹ is C₁-C₆-alkyl;
R² is C₁-C₆-alkyl or —(CH₂)ₙ—N(R⁵)₂;

### R³ is Cl or F;

### R⁴ is Cl or F;

each R⁵ is independently C₁-C₆-alkyl or, together with the nitrogen atom to which they are attached, form a morpholino, piperidinyl, or pyrazinyl group;
n is 2, 3, or 4;
p is 0 or 1; and
q is 0, 1, or 2.

In another aspect, n is 3.

In another aspect, p is 1.

In another aspect, q is 0 or 1.

In another aspect, the compound of formula A is represented by the following structure:

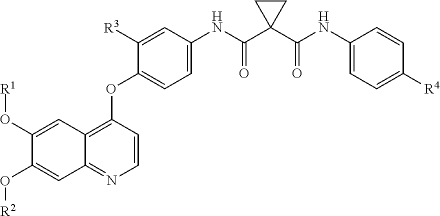

or a pharmaceutically acceptable salt thereof.

In another aspect, R¹ is methyl.

In another aspect, R³ and R⁴ are each F.

In another aspect, —CH₂)ₙ—N(R⁵)₂ is:

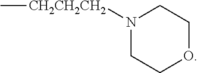

In another aspect, the compound of formula A is the compound of formula I (Compound I), represented by the following structure:

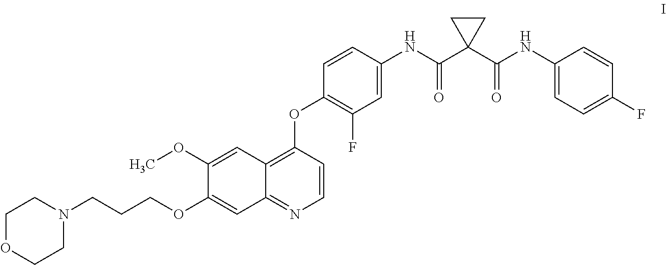 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt thereof.

In another aspect, the erbB inhibitor is a compound of formula II:

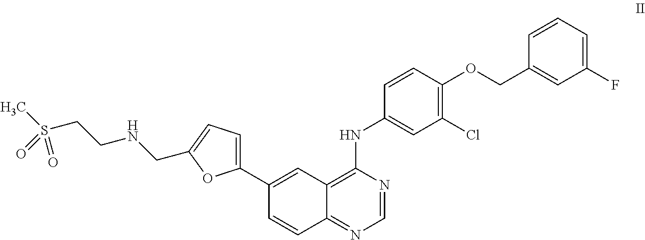 [SMILES: CS(=O)(=O)CCNCc1ccc(-c2ccc3ncnc(Nc4ccc(OCc5cccc(F)c5)c(Cl)c4)c3c2)o1]

or a pharmaceutically acceptable salt thereof. In another aspect, the erb inhibitor is a ditosylate salt or a ditosylate monohydrate salt of the compound of formula II.

In another aspect, the erbB inhibitor is a compound of formula III:

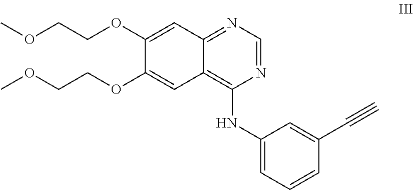 [SMILES: C#Cc1cccc(Nc2ncnc3cc(OCCOC)c(OCCOC)cc23)c1]

or a pharmaceutically acceptable salt thereof.

In another aspect the erbB inhibitor is trastuzumab (marketed under the name Herceptin).

In another aspect, the erbB inhibitor is cetuximab (marketed under the name Erbitux).

In another aspect, the erbB inhibitor is a monoclonal antihuman erbB3 antibody.

In another aspect, the erbB inhibitor is gefitinib (marketed under the name Iressa).

In another aspect, the cancer is gastric, lung, esophageal, head and neck, skin, epidermal, ovarian, or breast cancer.

In another aspect of the present invention there is provided a method of treating a patient suffering from breast cancer or head and neck cancer comprising administering to the patient a therapeutically effective amount of a compound of formula I, or a pharmaceutically acceptable salt thereof.

In another aspect of the present invention there is provided a method of treating a patient suffering from breast cancer or head and neck cancer comprising administering to the patient a therapeutically effective amount of a compound of formula I, or a pharmaceutically acceptable salt thereof.

In another aspect, a pharmaceutically acceptable excipient is included with the compound or pharmaceutically acceptable salt of formula A; or the erbB inhibitor; or a combination thereof.

As used herein, the term “effective amounts” means amounts of the drugs or pharmaceutical agents that will elicit the desired biological or medical response of a tissue, system, animal, or human. Furthermore, the term “therapeutically effective amounts” means any amounts which, as compared to a corresponding subject who has not received such amounts, results in improved treatment, healing, prevention, or amelioration of a disease, disorder, or side effect, or a decrease in the rate of advancement of a disease or disorder. The term also includes within its scope amounts effective to enhance normal physiological function. It is to be understood that the compounds can be administered sequentially or substantially simultaneously.

The method of the present invention can be administered by any suitable means, including orally or parenterally. Pharmaceutical formulations adapted for oral administration may be presented as discrete units such as capsules or tablets; powders or granules; solutions or suspensions in aqueous or non-aqueous liquids or oil-in-water liquid emulsions. The oral administration may include pharmaceutically acceptable excipients such as those known in the art.

Pharmaceutical formulations adapted for parenteral administration, especially intravenous administration, include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

As used herein, “an erbB inhibitor” refers to a compound, monoclonal antibody, immunoconjugate, or vaccine that inhibits erbB-1 or erbB-2 or or erbB-3 or a combination thereof.

The present invention includes compounds as well as their pharmaceutically acceptable salts. The word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, “patient” is a mammal, more particularly a human, suffering from cancer.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds of the method of the present invention herein may be prepared. These pharmaceutically acceptable salts may be prepared in situ during the final isolation and purification of the compound, or by separately reacting the purified compound in its free acid or free base form with a suitable base or acid, respectively.

In general, the dosing amount of the compound of Formula A and the erbB inhibitor is that amount which is both effective and tolerated. Preferably, the amount of the compound of Formula A, more particularly Compound I, is in the range of from about 1 mg to 1000 mg/day and the amount of the erbB inhibitor is preferably in the range of from about 1 μg to 2000 mg/day.

Compound I (N¹-{3-fluoro-4-[(6-(methyloxy)-7-{[3-(4-morpholinyl)propyl]oxy}-4-quinolinyl)oxy]phenyl}-N¹-(4-fluorophenyl)-1,1-cyclopropanedicarboxamide), can be prepared as described in WO2005/030140, published Apr. 7, 2005. Examples 25 (p. 193), 36 (pp. 202-203), 42 (p. 209), 43 (p. 209), and 44 (pp. 209-210) describe how Compound I can be prepared. Compounds of Formula A can be similarly prepared. The general preparation for Compound I is outlined in Scheme 1:

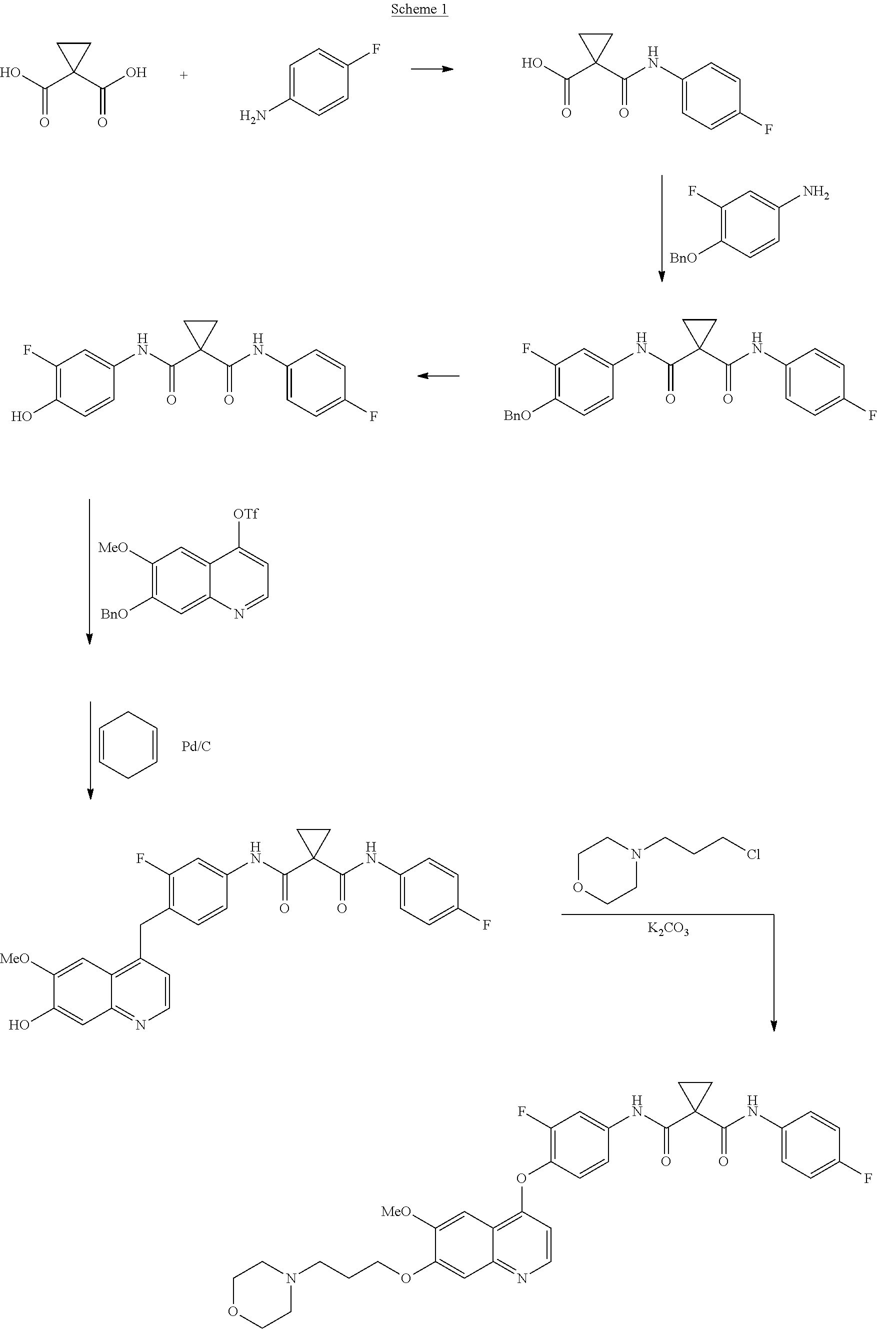

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine (represented by formula II, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

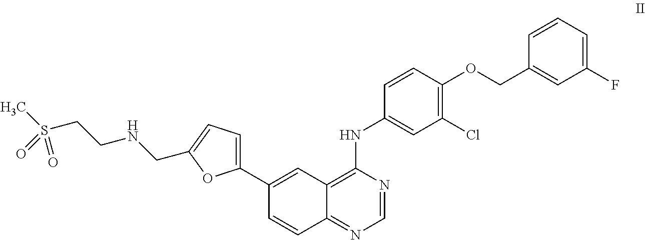 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (II) may be prepared according to the procedures disclosed in WO 99/35146, published Jul. 15, 1999; and WO 02/02552 published Jan. 10, 2002. The general scheme for the preparation of the ditosylate salt of Compound II is illustrated in Scheme 2.

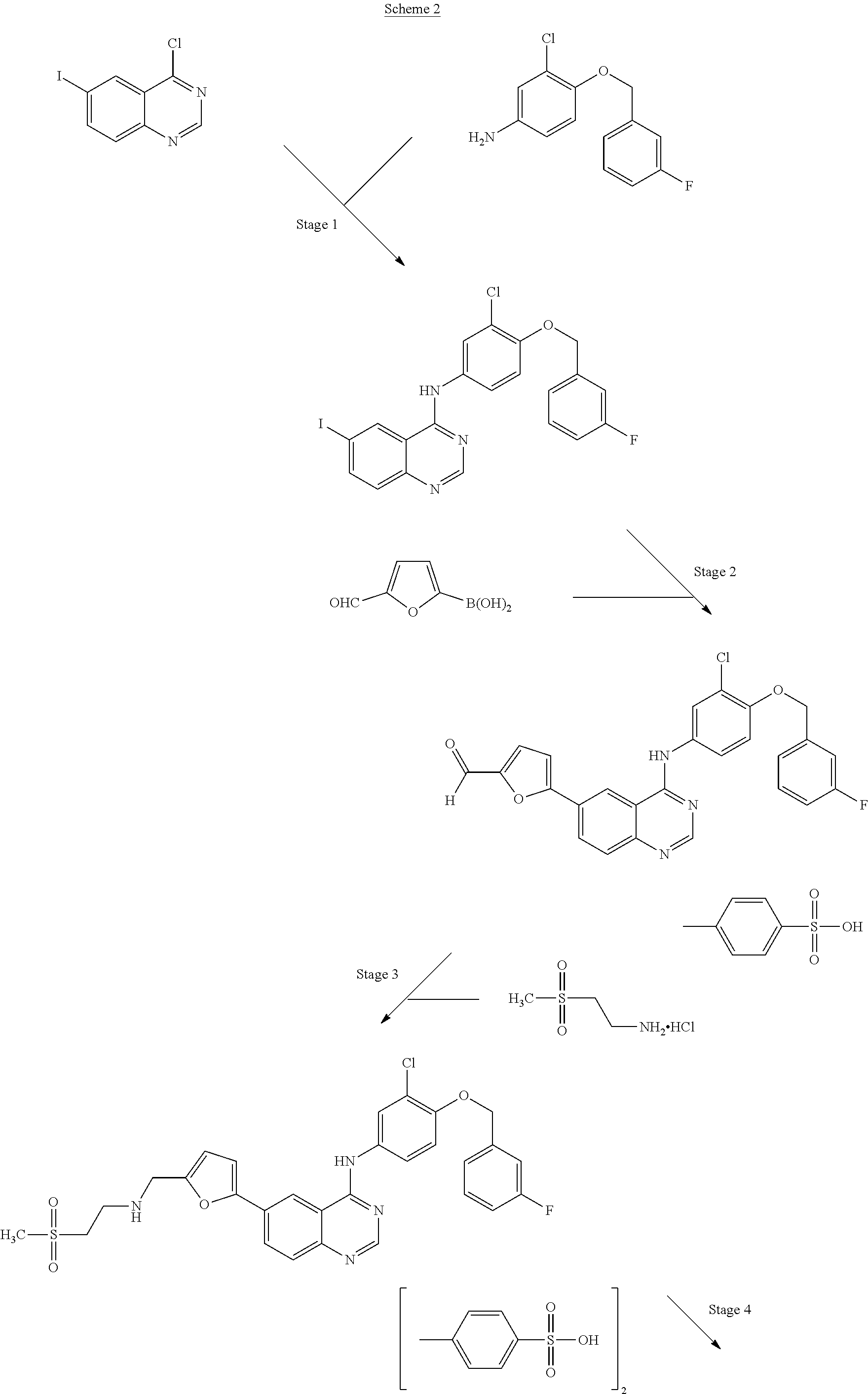

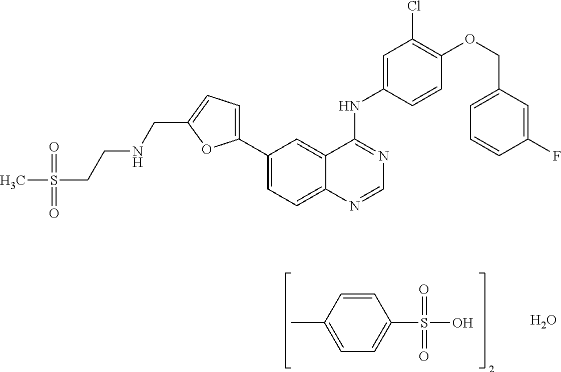

In Scheme 2, the preparation of the ditosylate salt of the compound of formula (I) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

Erlotinib, N-(3-ethynylphenyl)-6,7-bis{[2-(methyloxy)ethyl]oxy}-4-quinazolinamine (commercially available under the tradename Tarceva) is represented by formula III, as illustrated:

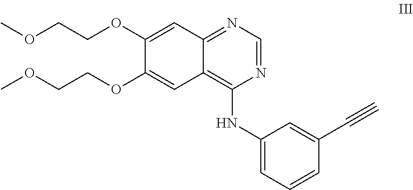 [SMILES: C#Cc1cccc(Nc2ncnc3cc(OCCOC)c(OCCOC)cc23)c1]

The free base and HCl salt of erlotinib may be prepared, for example, according to U.S. Pat. No. 5,747,498, Example 20.

Gefitinib, 4-quinazolinamine,N-(3-chloro-4-fluorophenyl)-7-methoxy-6-[3-4-morpholin)propoxy] is represented by formula IV, as illustrated:

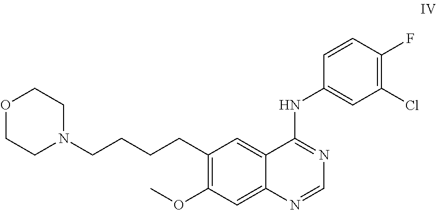 [SMILES: COc1cc2ncnc(Nc3ccc(F)c(Cl)c3)c2cc1CCCCN1CCOCC1]

Gefitinib, which is commercially available under the trade name IRESSA® (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

### Methods

### Cell Lines and Culture

Human breast carcinoma cell lines, BT474, HCC1954 and MDA-MB-468, head and neck squamous cell carcinoma lines, SCC15, Detroit 562 and SCC12, gastric carcinoma cell lines, SNU-5, HS746T, AGS, SNU-16 and N87, lung carcinoma cell lines, NCI-H1993, NCI-H1573, NCI-H441, NCI-H2342, NCI-H1648, HOP-92, NCI-H596, NCI-H69, NCI-H2170 and A549, epidermal carcinoma cell line A431, and colon carcinoma lines, HT29, SW48, and KM12 were purchased from the American Type Culture Collection (ATCC). Esophageal carcinoma cell line OE33 was purchased from ECACC European Collection of Cell Cultures (UK). Breast cancer cell line JIMT-1 and gastric carcinoma cell line MKN-45 were purchased from Deutsche Sammlung von Mikroorganismen and Zellkulturen GmbH (Germany); KPL-4, a human breast cancer cell line was kindly provided by Prof. J Kurebayashi (Kawasaki Medical School, Kurashiki, Japan). LL1-BT474-J4 (BT474-J4) breast carcinoma cell clone was developed by single-cell cloning of BT474 (HER2+ breast, highly sensitive to lapatinib) which had been exposed to increasing concentrations of lapatinib up to 3 μM. The LICR-LON-HN5 head and neck carcinoma cell line (HN5) was a gift from the Institute of Cancer Research, Surrey, U.K. HN5Cl2 was developed by single-cell cloning of HN5 followed by exposure to increasing concentrations of lapatinib.

BT474, HCC1954, MDA-MB-468, SCC15, Detroit 562, SCC12, SNU-5, HS746T, AGS, NCI-N87, A-431, NCI-H1993, NCI-H441, HOP-92, NCI-H596, NCI-H69, NCI-H2170, A549, JIMT-1, MKN-45, OE-33, SNU-16, SW48, KM12, and HT29 lines were cultured in a humidified incubator at 37° C. in 95% air, 5% CO₂ in the RPMI 1640 containing 10% fetal bovine serum (FBS) media. Both NCI-H1573, and NCI-H1648 were cultured in ACL-4 serum free medium containing 50:50 Dulbecco's modified Eagle medium (DMEM) /F12, Insulin Transferrin SeleniunX supplements, 50 nM Hydrocortisone, 1 ng/mL EGF, 0.01 mM ethanolamine, 0.01 mM phosphoryl-ethanolamine, 100 pM triiodothyronine, 0.5% (w/v) BSA (2 mg/mL), 2 L-glutamine, 0.5 mM sodium pyruvate. NCI-H2342 was cultured in the ATCC-formulated DMEM:F12 medium (Catalog No. 30-2006) with 0.005 mg/mL Insulin, 0.01 mg/mL Transferrin, 30 nM Sodium selenite (final conc.), 10 nM Hydrocortisone (final conc.), 10 nM beta-estradiol (final conc.), 10 nM HEPES (final conc.), extra 2 mM L-glutamine (for final conc. of 4.5 mM) and 5% fetal bovine serum (final conc.). BT474-J4 was cultured in RPMI 1640 containing 10% FBS and 1 μM lapatinib. KPL-4 and HN5 were cultured in DMEM containing 5% FBS; FINS Cl2 was cultured in DMEM containing 5% FBS and 1 μM lapatinib.

### Cell Growth Inhibition Assay and Data Analysis

Cell growth inhibition was determined via CellTiter-Glo cell viability assays. Cells were seeded in a 96-well tissue culture plate with the following plating densities in their respective media containing 10% FBS at 1000 or 2000 cells/well dependent on cell growth rate. BT474-J4 and HN5Cl2 were washed with PBS and plated in their culture media without lapatinib. Approximately 24 h after plating, cells were exposed to compounds; cells were treated with ten, two-fold serial dilutions (final compound concentrations ranging from 10, 5, 2.5, 1.25, 0.63, 0.31, 0.16, 0.08, 0.04 to 0.02 μM) of compound or the combination of the two agents at a constant molar to molar ratio of 1:1 or as indicated. Cells were incubated with the compounds in culture medium containing either 5% or 10% FBS and in the presence or absence of 2 ng/mL HGF, the ligand for cMET activation for 3 days, or as indicated. ATP levels were determined by adding Cell Titer Glo® (Promega), incubating for 20 minutes then the luminescent signal was read on the SpectraMax M5 plate with a 0.5 second integration time. Cell growth was calculated relative to vehicle (DMSO) treated control wells. The concentration of compound that inhibits 50% of control cell growth (IC₅₀) was interpolated using the following four parameter curve fitting equation:

y=(A+(B−A)/(1+10^{(x-c)d})

where A is the minimum response (yₘᵢₙ), B is the maximum response (yₘₐₓ), c is the inflection point of the curve (EC₅₀), d is the Hill coefficient, and x is the log₁₀ compound concentration (moles/L).

Combination effects were evaluated using both Combination Index (CI) values and Excess Over Highest Single Agent (EOHSA) statistical analysis.

CI values were calculated with the interpolated IC₅₀ values and the mutually non-exclusive equation derived by Chou and Talalay:

CI=Dₐ/IC₅₀₍ₐ₎+D_{b}/IC_{50(b)}+(Dₐ×D_{b})/(IC₅₀₍ₐ₎×IC_{50(b)})

where IC₅₀₍ₐ₎ is the IC₅₀ of the inhibitor A; IC_{50(b)} is the IC₅₀ for the inhibitor B; Dₐ is the concentration of the inhibitor A in combination with the inhibitor B that inhibited 50% of cell growth; and D_{b} is the concentration of inhibitor B in combination with the inhibitor A that inhibited 50% of cell growth. In general, a CI value between 0.9 and 1.10 indicates an additive effect for the combination of the two agents. A CI<0.9 indicates synergism (smaller number indicates a greater strength of synergy) and a CI>1.10 indicates antagonism.

Excess Over Highest Single Agent (EOHSA) is defined as a statistically significant improvement in the combination compared to the component monotherapies. For example, if compounds A and B are combined at concentrations q and r, respectively, then the average response in the combination Aq+Br will be significantly better than the average responses in Aq or Br alone. In statistical terms, the maximum of the p-value's for the two comparisons Aq+Br vs Aq and Aq+Br vs Br should be less than or equal to an appropriate cutoff, p≦0.05. EOHSA is a common approach for evaluating drug combinations, and is an FDA criterion (21 CRF 300.50) for combination drug approval. See Borisy et al. (2003) or Hung et al. (1993) for examples and discussion. Analysis was conducted using two-factor analysis of variance with interaction (model terms were dose of drug A, dose of drug B, and interaction between doses of drug A and B), followed by linear contrasts between each combination group and corresponding monotherapies. Analysis was conducted using SAS (version 9, provided by SAS Institute, Cary, N.C.). EOHSA at each dose was calculated as the minimum difference in average % inhibition between the combination and each monotherapy, from the appropriate ANOVA contrast. Since there are many comparisons for the % inhibition endpoint, p-value adjustment for multiple comparisons was performed. Hommel's procedure was implemented in order to improve the power while retaining Familywise Error Rate (FWE) control by using a sequentially rejective method. The p-values for both synergy and antagonism were calculated using this adjustment. Using the EOHSA method, synergy means that the effect (or response) in combination is significantly more than the highest single agent alone with p≦0.05; additive means that the effect in combination is not significantly different from the highest single agent alone (p>0.05), antagonist means that the effect in combination is significantly less than the highest single agent alone with p≦0.05.

### Cell Apoptosis Assays-Cell Death ELISA^{Plus} (Measuring DNA Fragmentation) and Caspase-Glo® 3/7 Assays

Cell apoptosis was measured by both a cell death ELISA method, which measures DNA fragmentation, a hallmark of apoptosis; and Caspase-Glo® 3/7 assay which detects the activity of capsase 3/7, one of the execution enzymes for apoptosis in cells.

The Cell Death ELISA^{Plus} kit (Roche, Mannheim, Germany) was used according to the manufacturer's instructions. Cells were seeded in 96-well plates at 10,000 per well. After 24 h, cells were dosed and grown for an additional 48 h in RPMI 1640 with 10% FBS in 5% CO₂ at 37° C. Cytoplasmic fractions of control and treated cells were transferred into streptavidin-coated 96-well plates and incubated with biotinylated mouse antihistone antibody and peroxidase-conjugated mouse anti-DNA antibody at room temperature for 2 h. Absorbance was determined at 405-490 nm using a Spectra Max Gemini microplate reader (Molecular Devices, Sunnyvale, Calif.).

The Caspase-Glo® 3/7 assay (Promega) is a homogeneous luminescent assay that measures caspase-3 and -7 activities. Cells were seeded in 96-well plates at 5,000 per well. After 24 h, cells were dosed and grown for an additional 24 h in RPMI 1640 with 10% FBS in 5% CO₂ at 37° C. Caspase 3/7 activity was detected by adding luminogenic caspase-3/7 substrate, which contains the tetrapeptide sequence DEVD, in a reagent optimized for caspase activity, luciferase activity and cell lysis, according to the manufacturer's instructions.

### Western Blot Analysis

Cells were plated at 250,000 to 500,000 per well in six-well plates (Falcon multiwell, Becton Dickinson, Franklin Lakes, N.J.). The following day, cells were treated with compounds in the growth medium containing 10% FBS. After treatment, cells were washed with cold PBS and lysed in the culture dishes using cell lysis buffer [40 mmol/L Tris-HCl (pH 7.4), 10% glycerol, 50 mmol/L beta-glycerophosphate, 5 mmol/L EGTA, 2 mmol/L EDTA, 0.35 mmol/L vanadate, 10 mmol/L NaF, and 0.3% Triton X-100] containing protease inhibitors (Complete Protease Inhibitor Tablets, Boehringer Mannheim, Indianapolis, Ind.). The protein samples (50 μg), determined using Bio-Rad detergent-compatible protein assays, from control and treated cell lysates were loaded on 4% to 12% gradient NuPAGE gels (Novex, Inc., San Diego, Calif.), electrophoresed under reducing conditions, and transferred onto nitrocellulose membranes (0.45 μm; Bio-Rad Laboratories). The membrane blots were rinsed with PBS and blocked in Odyssey Blocking buffer for 1 h at RT. Blots were probed with antibodies against specific proteins in blocking buffer plus 0.1% Tween 20 and incubated for 2 h at room temperature. The membranes were washed and incubated with IRDye 680 or IRDye 800 secondary antibodies at RT for 1 h in blocking buffer plus 0.1% Tween 20. The membranes were developed with Odyssey Infrared Imaging System (LI-COR Biosciences, Lincoln, Nebr.).

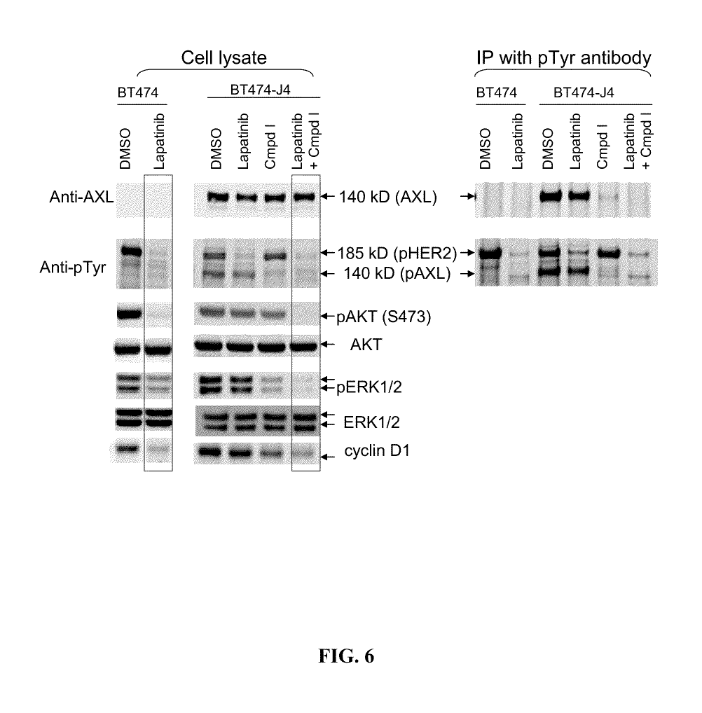

The conditions used for the western blot analysis (FIG. 6) were as follows: Cells were treated with lapatinib (1 μM) alone, Compound I alone (1 μM), or lapatinib (1 μM) in combination with Compound I (1 μM), for 4 h. Cell lysate (50 μg of total protein) or proteins immunoprecipitated with anti-phospho-Tyrosine antibody were loaded in SDS-PAGE gel. The antibody against specific protein was used in the western blot analysis.

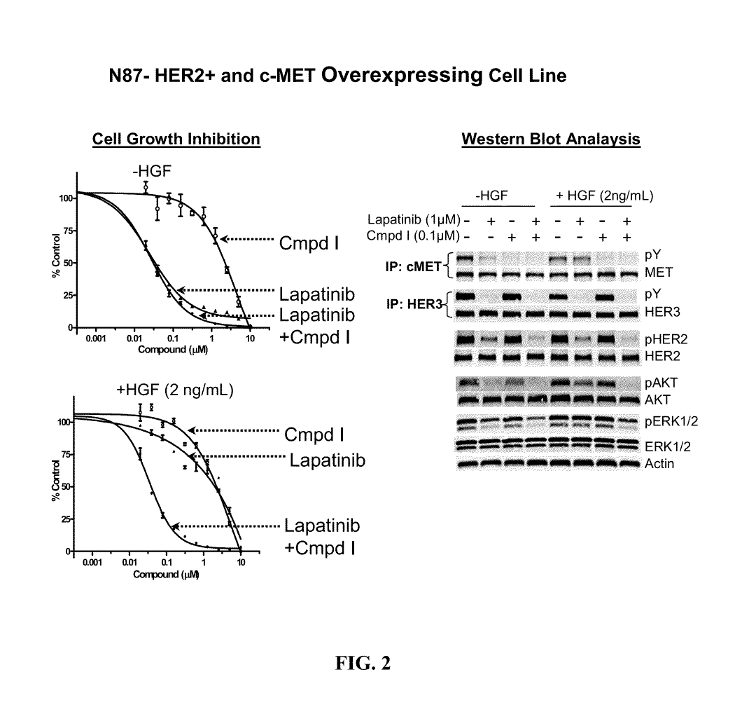

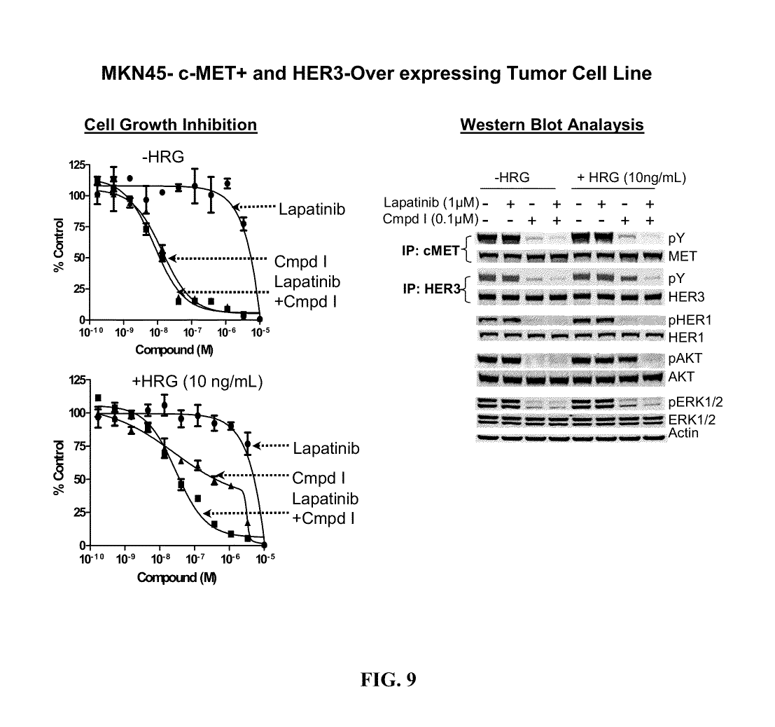

The conditions used for the western blot analysis (FIG. 2 right panel and FIG. 9 right panel) were as follows: Cells were treated with lapatinib (1 μM) alone, Compound I alone (0.1 μM), or lapatinib (1 μM) in combination with Compound I (0.1 μM), for 2 h in the absence or presence of HGF or HRG as indicated. Cell lysate (50 μg of total protein) or proteins immunoprecipitated with anti-MET or anti-HER3 antibody were loaded in SDS-PAGE gel. The antibody against specific protein was used in the western blot analysis.

### Compound I Cell Growth Inhibition

Compound I is a potent multi kinase inhibitor that targets cMET, RON, AXL, VEGFR 1/2, TIE2, PDGFRbeta, cKIT and FLT3. Cell growth inhibition was determined via CellTiter-Glo cell viability assay in breast (BT474, HCC1954, KPL-4, JIMT-1, MDA-MB-468 and BT474-J4), head and neck (SCC15, FINS, Detriot 562, SCC12 and HN5Cl2), gastric (SNU-5, MKN-45, HS746T, AGS, SNU-16 and NCI-N87), lung (NCI-H1993, NCI-H1573, NCI-H441, NCI-H2342, NCI-H1648, HOP-92, NCI-H596, NCI-H69, NCI-H2170, A549), esophageal (OE-33), skin (A431) and colon (HT29, SW48 and KM12) tumor cell lines.

Hepatocyte growth factor (HGF) is the ligand for cMET activation. It is a cytokine with several biological activities, including stimulation of cell proliferation, motility, and morphogenesis. HGF is secreted as an inactive precursor that is converted to the active heterodimeric form by secreted proteases, including plasminogen activators. In in vitro cell culture conditions, most of tumor cell lines do not express the active form of HGF. Adding the active form of human HGF to the culture medium provides a paracrine cMET activation system. HGF level from human serum was reported in healthy humans to be ˜0.2 ng/mL (J. Immunol. Methods 2000; 244:163-173) and increased up to 2 ng/mL in liver metastasis breast cancer patients (Tumor Biol 2007; 28:36-44). Therefore, HGF was added at 2 ng/mL to the culture medium containing either 5% or 10% FBS for the cell growth inhibition and apoptosis assays.

### Abbreviations for Tables

The following is an explanation of the abbreviations used in the tables:

N=2 means that the experiments are repeated two times independently. All of the analyses were carried out in duplicate except where indicated with an asterisk;
IC50 means the concentration of compound that inhibits 50% of control cell growth interpolated using the four parameter curve fitting equation, μM refers to micromoles per liter;
HER amp+ indicates that gene HER1 (HER1+), or HER2 (HER2+) is amplified in the cell line; “no” means that neither HER1 nor HER2 is amplified in the cell line;
>10 means that an IC₅₀ was not achieved up to the highest concentration tested (10 μM);
HER3-over refers to over-expression levels of HER3RNA (MAS 5 intensitiy>300) as determined by Affymetrix microarray analyses;
HER3-over refers to over-expression levels of HER3RNA (MAS 5 intensitiy<100) as determined by Affymetrix microarray analyses; cMET+ refers to cMET gene amplification with ≧5 copies of MET DNA as determined by SNP-CHIP;
cMET+ (<5) refers to cMET gene amplification with <5 copies of MET DNA as determined by SNP-CHIP;
cMET-over refers to over-expression of cMET RNA (MAS 5 intensity >300) as determined by Affymetrix microarray analyses;
cMET-low refers to low expression levels of cMET RNA (MAS 5 intensity <300) as determined by Affymetrix microarray analyses;
cMET-mut refers to a point mutation, deletion, insertion or missense mutation in cMET gene;
−HGF means that no HGF was added;
+HGF means that 2 ng/mL HGF was added to the culture medium containing 5% or 10% FBS.
−HRG means that no HRG was added;
+HRG means that 10 ng/mL HRG was added to the culture medium containing 10% FBS.
NA=not applicable because the absolute IC₅₀ value of the agent alone could not be determined.

### Cell Growth Inhibitory Effects of Compound I

The growth inhibitory effects of Compound I alone in the tumor cell lines are summarized in Table 1. As Table 1 shows, this compound is very potent at inhibiting cell growth for the cMET+ and HER non-amplified (HER+=no) tumor lines MKN-45, SNU-5, HS746T, and NCI-H1993, exhibiting IC₅₀ values of less than 100 nM. NCI-H1648, a cMET amplified lung tumor cell line, is more sensitive to Compound I in the presence of HGF, suggesting a HGF-cMET activation dependent cell growth of this line.

TABLE 1
IC₅₀ (μM) values of cell growth inhibition
by Compound I alone in tumor cell lines.

| | | | Cmpd I | |
|---|---|---|---|---|
| | | HER | (IC₅₀, μM), N = 2 | |
| Cell lines | cMET | amp+ | −HGF | +HGF |
| gastric_SNU-5 | cMET+ | no | 0.012 | 0.019 |
| gastric_MKN-45 | cMET+ | no | 0.014 | 0.019 |
| lung_H1993 | cMET+ | no | 0.044 | 0.087 |
| gastric_HS746T | cMET+ | no | 0.044 | 0.162 |
| lung_H1648 | cMET+ | no | 1.202 | 0.470 |
| eso_OE33 | cMET+ | HER2+ | 0.386 | 0.445 |
| lung_H1573 | cMET+ | HER1+ | 1.651 | 1.478 |
| hn_Detroit562 | cMET+ (<5) | no | 0.458 | 0.450 |
| lung_H441 | cMET+ (<5) | no | 1.031 | 1.155 |
| lung_H2342 | cMET+ (<5) | no | 1.925 | 1.452 |
| lung_H596 | cMET-mut(E14Del) | no | 1.061 | 0.705 |
| lung_H69 | cMET-mut(R988C) | no | 1.274 | 0.970 |
| lung_HOP-92 | cMET-mut(T1010I) | no | 0.827 | 0.566 |
| gastric_SNU16 | cMET-over | no | 0.055 | 0.054 |
| lung_A549 | cMET-over | no | 0.885 | 0.411 |
| colon_HT-29 | cMET-over | no | 0.556 | 0.559 |
| colon_SW48 | cMET-over | no | 0.260 | 0.220 |
| colon_KM12 | cMET-over | no | 0.040 | 0.100 |
| lung_H2170 | cMET-over | HER2+ | 0.684 | 0.522 |
| skin_A431 | cMET-over | HER1+ | 0.687 | 0.674 |
| hn_SCC15 | cMET-over | HER1+ | 0.700 | 0.690 |
| hn_HN5 | cMET-over | HER1+ | 0.726 | 0.824 |
| hn_SCC12 | cMET-over | no | 0.988 | 1.189 |
| hn_HN5C2 | cMET-over | HER1+ | 0.858 | 1.213 |
| breast_HCC1954 | cMET-over | HER2+ | 1.855 | 1.856 |
| breast_JimT1 | cMET-over | HER2+ | 1.732 | 1.911 |
| gastric_N87 | cMET-over | HER2+ | 2.446 | 2.320 |
| breast_KPL4 | cMET-low | HER2+ | 0.459 | 0.625 |
| gastric_AGS | cMET-low | no | 0.656 | 0.427 |
| breast_MDA-MB-468 | cMET-low | HER1+ | 0.813 | 0.589 |
| breast_BT474-J4 | cMET-low | HER2+ | 4.515 | 4.016 |
| breast_BT474 | cMET-low | HER2+ | 4.974 | 4.899 |

The results from Table 1 indicate that tumor cells with cMET gene amplification are highly dependent on cMET for proliferation. As Table 1 further illustrates, Compound I showed IC₅₀ values ranging from 0.04 to ˜5 μM in cell growth inhibition in cell lines with cMET amplification of less than 5 copies, with cMET mutations at the juxtamembrane domain (HOP-92: cMET-T1010I; H69: cMET-R988C and H596: cMET-exon 14 in frame deletion) or cMET non-amplified tumor lines which express high or low amounts of cMET RNA, designated cMET over and cMET-low, respectively. These results are consistent with the observation that Compound I inhibits multiple oncogenic kinases in tumor cells.

Cell Growth Inhibition Effect of Compound I in Combination with Lapatinib on Cell Lines with cMET and HER Amplification

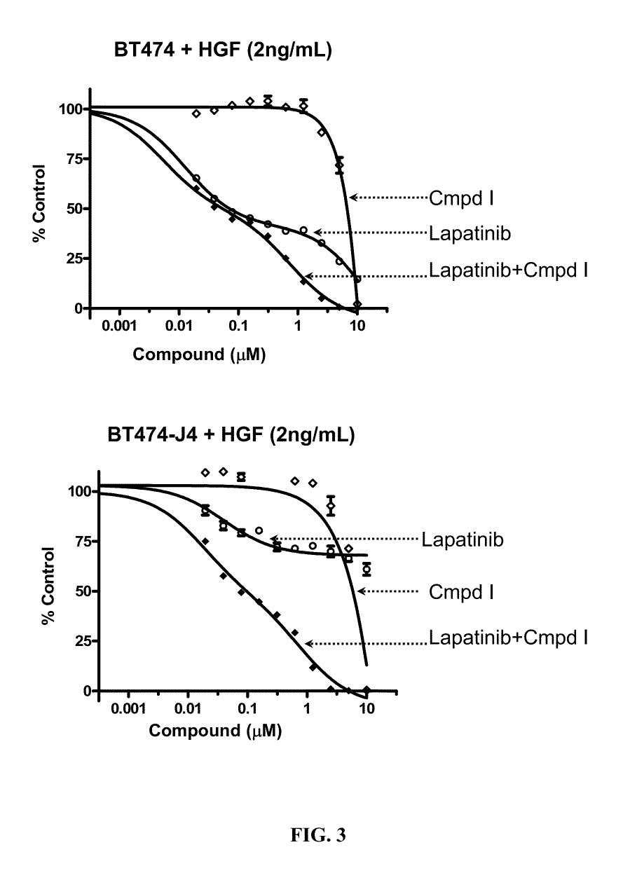

As illustrated in Table 2, lapatinib alone exhibited average IC₅₀s of 0.12 and 0.11 (with and without HGF respectively) in breast BT474 tumor cell line with low cMET and HER2+ while Compound I alone exhibited average IC₅₀s of 4.97 μM (with HGF) and 4.90 μM (without HGF). This result is not surprising since lapatinib, unlike Compound I, is known to be a potent inhibitor of amplified erbB-2 (HER amp+). In combination, lapatinib and Compound I showed either an additive effect based on CI of 0.95 without HGF or a synergistic effect based on CI of 0.71 with HGF, and enhanced cell growth inhibition at higher concentrations (FIG. 3) in breast_BT474 cell line.

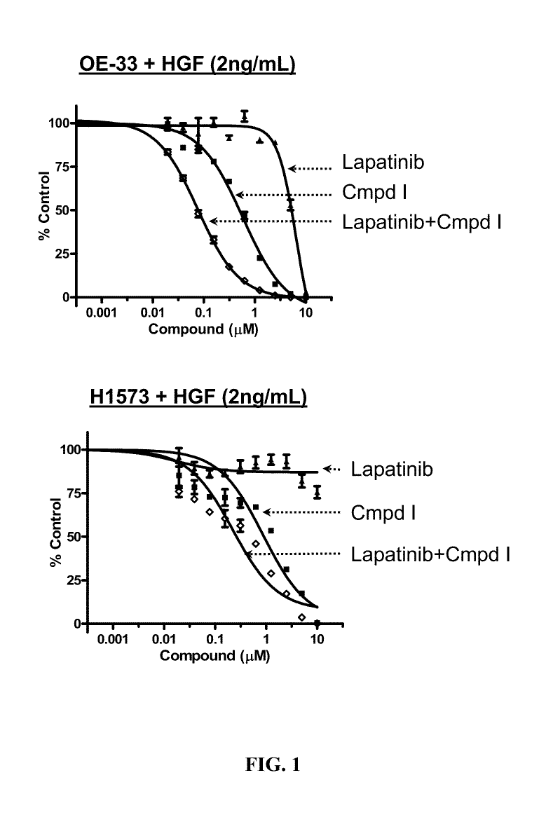

In comparison, the effect of cell growth inhibition in an esophageal tumor cell line with co-amplified cMET and HER2 (eso_OE33) by the combination of lapatinib and Compound I is remarkable and unexpected. As Table 2 and FIG. 1 shows, OE33 showed resistance to lapatinib (IC₅₀=6.5 μM without HGF, >10 μM with HGF) and was moderately sensitive to Compound I (IC₅₀=0.42 μM without HGF, 0.40 μM with HGF) alone. However, the combination of lapatinib with Compound I showed robust synergistic effect (based on both CI and EOHSA) of cell growth inhibition in OE-33 esophageal tumor cells with and without HGF. Similarly, as shown in Table 2 and FIG. 1, NCI-H1573, a lung tumor cell line with cMET and EGFR co-amplification is resistant to lapatinib and moderately sensitive to Compound I if administrating separately; however, the combination of the two inhibitors improved the potency (lower the IC₅₀ values) and increased cell growth inhibition activity (synergy based on EOHSA). Though not bound by theory, these results suggest that cMET and HER can interact (“cross-talk”) and escape the growth inhibition provided by a HER inhibitor or cMET inhibitor alone, and that the combination of lapatinib with Compound I overcomes the resistance in cMET and HER co-amplified tumor cells.

TABLE 2
Cell growth inhibition effect of Compound I and lapatinib combination on tumor
cell lines with co-amplification of both cMET and HER1 or HER2 genes.

| | | | Average IC50 (μM) N = 2 | | | | | | | |
|---|---|---|---|---|---|---|---|---|---|---|
| | | | | | Lapatinib | | | | | |
| | | | | | or Cmpd I | | | | Combination | |
| | | | | | (Lapatinib + | | | | Effect | |
| | | HER | Lapatinib | | Cmpd I) | | Cmpd I | | CI @IC50 | |
| Cell lines | cMET | amp+ | −HGF | +HGF | −HGF | +HGF | −HGF | +HGF | −HGF | +HGF |
| eso_OE33 | cMET+ | HER2+ | 6.52 | 5.52 | 0.04 | 0.07 | 0.42 | 0.40 | 0.11 | 0.20 |
| lung_H1573 | cMET+ | HER1+ | 9.83 | >10 | 0.52 | 0.41 | 1.52 | 1.38 | 0.41 | NA |
| breast_BT474 | cMET-low | HER2+ | 0.11 | 0.11 | 0.10 | 0.07 | 4.76 | 4.72 | 0.93 | 0.72 |

Cell Growth Inhibition Effect of Compound I in Combination with Lapatinib on Tumor Cell Lines with cMET Amplification, Mutation or Over-Expression

As shown in Table 3, the combination of lapatinib and Compound I showed synergistic effects with CI<0.9 in the cMET amplified, mutated, or over-expressed breast, lung, gastric, head and neck, ovarian, and skin tumor cells. EOHSA analysis confirmed synergy in all cases except N87 without HGF and H1993 with or without HGF. In each of these exceptions, single agent lapatinib or Compound I was very active by itself and the combination effect was additive.

Surprisingly, as shown in Table 3, HGF reduced the potency of cell growth inhibition by lapatinib in HER1/HER2 amplified and cMET over-expressed tumor cells (HER2+: N87, H2170, and HCC1954; HER1+: SCC15, HN5, and A431). Furthermore, combining lapatinib with Compound I not only overcame the HGF effect, but also increased sensitivity, especially in cell lines H2170, HCC1954, SCC15, HN5, and A431 with and without HGF. In contrast, HGF did not reduce the lapatinib activity in BT474 (Table 2) and KPL-4 (Table 3), two HER2 amplified breast tumor cell lines with low expression of cMET RNA or protein expression.

The HGF effect is illustrated in FIG. 2 for N87. FIG. 2 (left panel, labeled Cell Growth Inhibition) shows that in the absence of HGF, N87 was highly sensitive to lapatinib alone (IC₅₀=0.05 μM) or in combination at a 1:1 molar-to-molar ratio with Compound I. In contrast, in the presence of HGF, N87 is insensitive to lapatinib (IC₅₀=4.80 μM) but quite sensitive to the combination of lapatinib and Compound I (IC₅₀=0.05 μM). FIG. 2 (right panel, labeled Western Blot Analysis) also shows that the combination of lapatinib and Compound I inhibits phosphorylation of HER2, HER3 and cMET, and decreases the cell signaling of pAKT and pERK, consistent with the cell growth inhibition in both the presence and absence of HGF.

Table 3 and FIG. 2 are consistent with previous discoveries that support the claim that HGF activates cMET. The above results further suggest that HGF-mediated cMET activation may interact with HER and reduce the growth inhibition by a HER inhibitor. These results demonstrate that combining Compound I with lapatinib may provide a more effective therapy in cMET over-expressed and HER amplified tumor cells.

TABLE 3
Cell growth inhibition effect of compound I and lapatinib combination on tumor
cell lines with cMET amplification, mutation or over-expression.

| | | | Average IC50 (μM) N = 2 | | | | | | | |
|---|---|---|---|---|---|---|---|---|---|---|
| | | | | | Lapatinib | | | | | |
| | | | | | or Cmpd I | | | | Combination | |
| | | | | | (Lapatinib + | | | | Effect | |
| | | HER | Lapatinib | | Cmpd I) | | Cmpd I | | CI @IC50 | |
| Cell lines | cMET | amp+ | −HGF | +HGF | −HGF | +HGF | −HGF | +HGF | −HGF | +HGF |
| gastric_N87 | cMET-over | HER2+ | 0.05 | 4.80 | 0.04 | 0.05 | 2.62 | 2.62 | 0.77 | 0.03 |
| lung_H2170 | cMET-over | HER2+ | 0.26 | 4.24 | 0.12 | 0.08 | 0.68 | 0.50 | 0.79 | 0.19 |
| breast_HCC1954 | cMET-over | HER2+ | 0.80 | 5.27 | 0.12 | 0.25 | 1.85 | 1.98 | 0.47 | 0.18 |
| breast_KPL4 | cMET-low* | HER2+ | 1.00 | 0.89 | 0.10 | 0.11 | 0.64 | 0.35 | 0.33 | 0.51 |
| ovary_SKOV3 | cMET-over | HER2+ | 5.02 | 5.67 | 0.58 | 0.51 | 1.57 | 1.43 | 0.53 | 0.48 |
| hn_SCC15 | cMET-over | HER1+ | 1.08 | 3.81 | 0.13 | 0.16 | 0.66 | 0.66 | 0.33 | 0.29 |
| skin_A431 | cMET-over | HER1+ | 2.19 | 4.60 | 0.27 | 0.24 | 0.69 | 0.65 | 0.55 | 0.44 |
| hn_HN5 | cMET-over | HER1+ | 2.37 | 3.69 | 0.20 | 0.23 | 0.88 | 0.97 | 0.33 | 0.32 |
| hn_SCC12 | cMET-over | no | >10 | >10 | 0.30 | 0.37 | 1.11 | 1.16 | NA | NA |
| lung_H1993 | cMET+ | no | >10 | >10 | 0.01 | 0.02 | 0.02 | 0.09 | NA | NA |
| lung_H1648 | cMET+ | no | 7.39 | >10 | 0.15 | 0.06 | 1.18 | 0.52 | 0.15 | NA |
| hn_Detroit562 | cMET+ (<5) | no | 4.02 | 4.64 | 0.15 | 0.17 | 0.41 | 0.41 | 0.44 | 0.44 |
| lung_H2342 | cMET+ (<5) | no | 6.81 | 6.64 | 0.65 | 0.55 | 1.80 | 1.52 | 0.50 | 0.47 |
| lung_H441 | cMET+ (<5) | no | >10 | >10 | 0.67 | 0.63 | 1.12 | 1.17 | NA | NA |
| lung_H596 | cMET-mut | no | >10 | >10 | 0.67 | 0.43 | 1.18 | 0.82 | NA | NA |
| lung_H69 | cMET-mut | no | 5.36 | 4.74 | 0.72 | 0.61 | 1.27 | 0.97 | 0.78 | 0.83 |
| lung_HOP-92 | cMET-mut | no | >10 | >10 | 0.44 | 0.33 | 0.83 | 0.57 | NA | NA |

*Based on protein expression.

### The Combination Effects of Compound I and Lapatinib on Lapatinib Resistant HER+Tumor Cell Lines.

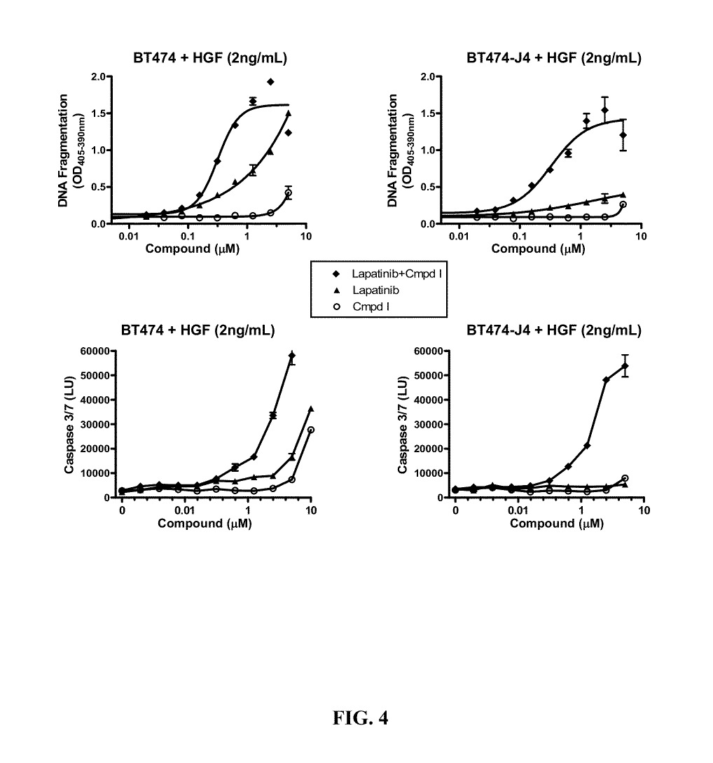

BT47444, JIMT1, and HN5Cl2 are lapatinib resistant HER2+ or HER1+ cell lines. JIMT-1, an inherited resistant line to lapatinib or trastuzumab, was derived from a patient who did not respond to trastuzumab. Both BT474-J4 and HN5Cl2 are lapatinib acquired resistant clones. As Table 4 shows, the combination of Compound I with lapatinib shows synergy (by EOHSA analysis) of cell growth inhibition in all three lapatinib resistant tumor cell lines. Moreover, as shown in FIG. 3, Compound I restores lapatinib sensitivity in the resistant BT474-J4 cells and increased lapatinib activity in both BT474 (sensitive to lapatinib) and BT474-J4 (resistant to lapatinib and trastuzumab) cells. The synergistic effect of Compound I and lapatinib in combination was not only detected in cell growth inhibition, but also in apoptosis induction as illustrated in FIG. 4. As FIG. 4 shows, combining Compound I and lapatinib increased both DNA fragmentation and caspase 3/7 activation, hallmarks of apoptosis, in both BT474 and BT474-J4 cells; however, administered separately, Compound I at high concentration or lapatinib induces apoptosis only in BT474, the lapatinib sensitive line.

TABLE 4
Cell growth inhibition effect of Compound I in combination with lapatinib on
lapatinib resistant HER+ tumor cell lines.

| | | | Average IC50 (μM) N = 2 | | | | | | | |
|---|---|---|---|---|---|---|---|---|---|---|
| | | | | | Lapatinib | | | | | |
| | | | | | or Cmpd I | | | | Combination | |
| | | | | | (Lapatinib + | | | | Effect | |
| | | HER | Lapatinib | | Cmpd I) | | Cmpd I | | CI @IC50 | |
| Cell lines | cMET | amp+ | −HGF | +HGF | −HGF | +HGF | −HGF | +HGF | −HGF | +HGF |
| breast_BT474 | cMET-low | HER2+ | 0.11 | 0.11 | 0.10 | 0.07 | 4.76 | 4.72 | 0.93 | 0.72 |
| breast_BT474-J4 | cMET-low | HER2+ | >10 | >10 | 0.08 | 0.07 | 4.79 | 4.05 | NA | NA |
| breast_JimT1 | cMET-over | HER2+ | >10 | >10 | 0.73 | 0.74 | 1.77 | 2.19 | NA | NA |
| hn_HN5Cl2 | cMET-over | HER1+ | 4.12 | 3.85 | 0.31 | 0.41 | 0.81 | 1.26 | 0.50 | 0.47 |

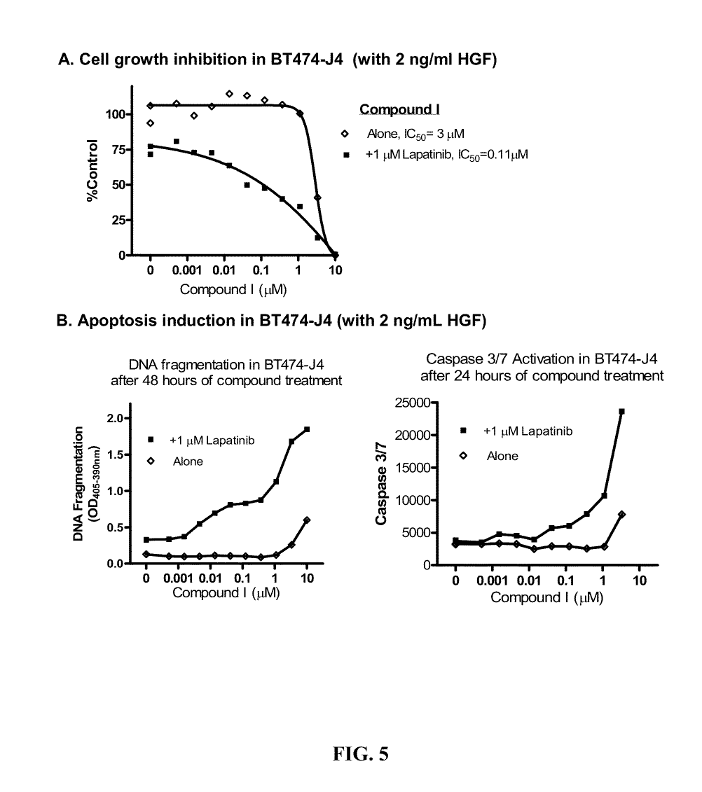

The dose responses of Compound I in cell line BT474-J4 were determined using a fixed concentration of lapatinib at 1 μM. As FIG. 5A shows, the IC₅₀ of Compound I was found to be 0.11 μM at a lapatinib concentration of 1 μM. Without lapatinib, the IC₅₀ of Compound I was 3 μM, while lapatinib by itself at 1.0 μM showed minimal effect (<50% inhibition). Further, as shown in FIG. 5B, an apoptosis induction was also detected when Compound I and lapatinib were combined under the same dosing conditions.

### Restoration of Lapatinib Sensitivity by Compound I Inhibition of AXL in BT474-J4 Cells

AXL was unexpectedly found to be highly expressed and phosphorylated in BT474-J4, but not expressed in BT474 cells, as determined by western blot analysis (illustrated in FIG. 6) and confirmed by quantitative RT-PCR. AXL has been reported to be overexpressed in several cancers including colon (Craven et al., Int J Cancer 1995; 60:791-7), lung (Shieh et al., Neoplasia 2005; 7:1058-64), esophageal (Nemoto et al., Pathobiology. 1997; 65(4):195-203), thyroid (Ito et al., Thyroid 1999, 9(6):563-7), ovarian (Sun et al, Oncology 2004; 66:450-7), gastric (Wu et al, Anticancer Res. 2002; 22(2B):1071-8), and breast cancer (Berclaz et al., Ann Oncol 2001; 12:819-24), where it is associated with poor prognosis. Overexpression of AXL in tissue culture causes oncogenic transformation. Accordingly, the combination of the present invention is useful for the treatment of all of these AXL-overexpressed tumors.

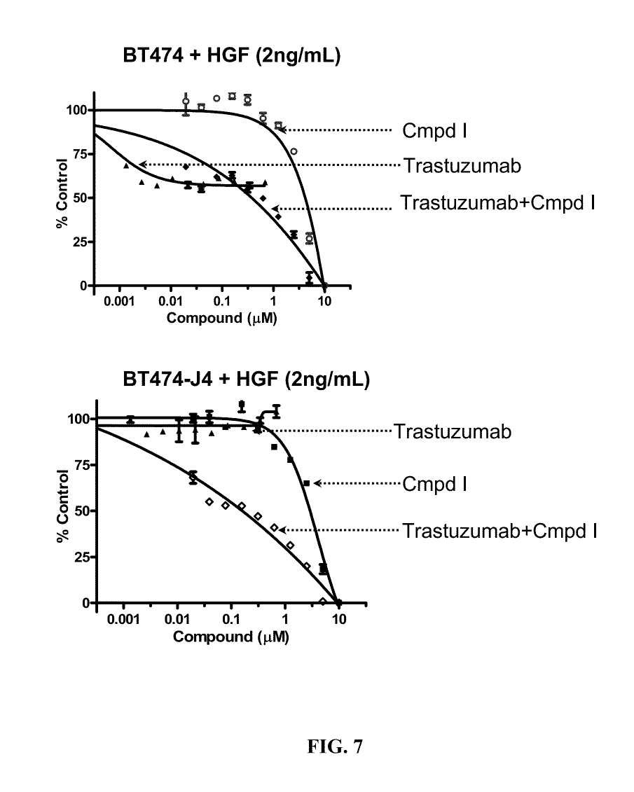

As FIG. 6 further shows, lapatinib alone inhibits phosphorylation of HER2 in both BT474 and BT474-J4 cells; however, lapatinib inhibits the downstream signaling of phosphorylation of AKT and ERK and reduces the level of cyclin D1 only in BT474, but not in BT474-J4 cells. On the other hand, Compound I alone inhibits the phosphorylation of AXL, but not the downstream signaling of phosphorylation of AKT in BT474-J4 cells. Surprisingly, the combination of Compound I and lapatinib substantially inhibits the phosphorylation of HER2, AXL, AKT, and ERK and decreases cyclin D1 level in BT474-J4 cells. The above cell signaling inhibition effect correlates very well with the robust synergy detected with combination of Compound I and lapatinib in cell growth inhibition and apoptosis induction in BT474-J4. These results, as well as the results shown in Table 5 and FIG. 7, provide evidence that 1) AXL over-expression confers a resistant mechanism to lapatinib or trastuzumab, and 2) the combination of Compound I and lapatinib or trastuzumab overcame the resistance in these tumor cells.

### Effect of Compound I and Trastuzumab Combination on HER2+ Tumor Cell Line.

Trastuzumab is a humanized monoclonal antibody which binds to the extracellular segment of the HER2 receptor and inhibits HER2 signaling. As illustrated in FIG. 7, trastuzumab alone showed 40% (without HGF) and 35% (with HGF) cell growth inhibition in BT474 cells, and no significant inhibition in BT474-J4, OE-33 and N87 cells after 5 days of treatment. As shown in Table 5, the combination of Compound I with trastuzumab increased cell growth inhibition in all four HER2 amplified lines as indicated by a lower IC₅₀ value or synergy using EOHSA analysis. The results further demonstrate the benefit of combining Compound I with a HER2 inhibitor in HER2 amplified tumor cell lines.

TABLE 5
Cell growth inhibition effect of compound I and trastuzumab on HER2+ tumor cell lines.

| | | | | IC50 (μM), 5 day treatment, N = 2 | | | |
|---|---|---|---|---|---|---|---|
| | | | | | Trastuzumab in | Cmpd I in | |
| Cell Lines | cMET | HER+ | HGF | Trastuzumab | (Trastuzumab + Cmpd I) | (Trastuzumab + Cmpd I) | Cmpd I |
| breast_BT474 | cMET-low | HER2+ | −HGF | >0.687** | 0.032 | 0.465 | 3.651 |
| breast_BT474 | cMET-low | HER2+ | +HGF | >0.687** | 0.051 | 0.746 | 3.941 |
| breast_BT474_J4 | cMET-low | HER2+ | −HGF | >0.687 | 0.010 | 0.139 | 2.954 |
| breast_BT474_J4 | cMET-low | HER2+ | +HGF | >0.687 | 0.009 | 0.132 | 2.909 |
| gastric_N87 | cMET-over | HER2+ | −HGF | >0.687 | 0.039 | 0.570 | 1.361 |
| gastric_N87 | cMET-over | HER2+ | +HGF | >0.687 | 0.040 | 0.582 | 1.616 |
| eso_OE33 | cMET+ | HER2+ | −HGF | >0.687 | 0.001 | 0.016 | 0.035 |
| eso_OE33 | cMET+ | HER2+ | +HGF | >0.687 | 0.003 | 0.037 | 0.127 |

**Trastuzumab inhibited 35~40%% cell growth maximally in BT474 after 5 days of treatment.

### Effect of Compound I and Erlotinib on Tumor Cell Lines.

Erlotinib is an EGFR inhibitor and at high concentrations also inhibits HER2 in cell culture. Erlotinib alone was not very active in most of the tumor cell lines tested. Combination of Compound I and erlotinib showed synergy of cell growth inhibition as indicated as CI<0.9 and confirmed by EOHSA analysis in the lung, head and neck, breast, ovarian, gastric, and epidermal tumor cell lines listed in Table 6.

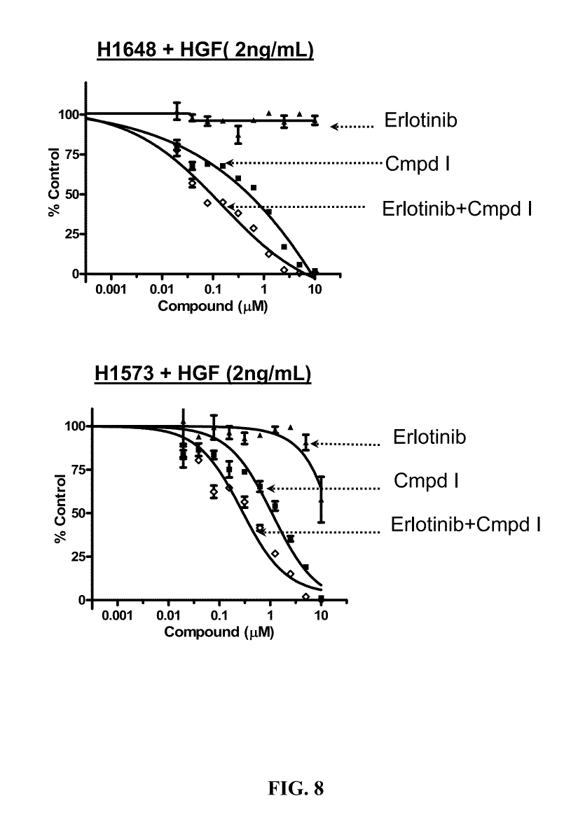

Notably, as illustrated in FIG. 8, NCI-H1648 lung tumor cell line was found to be resistant to erlotinib (IC₅₀>10 μM) and moderately sensitive to Compound I (IC₅₀=0.96 μM without HGF, 0.40 μM with HGF), but highly sensitive to the combination of erlotinib and Compound I. Similarly, NCI-H1573, a lung tumor cell line with cMET and EGFR co-amplification was found to be resistant to erlotinib and moderately sensitive to Compound I but was more sensitive to the combination of the two compounds. These results suggest that combining erlotinib with Compound of formula I could provide more effective treatment in these tumor cells.

TABLE 6
Cell growth inhibition effect of Compound I and erlotinib combination on breast,
colon, gastric, head and neck, lung, ovarian, and skin tumor cell lines.

| | | | Average IC50 (♦M) N = 2 | | | | | | | |
|---|---|---|---|---|---|---|---|---|---|---|
| | | | | | Erlotinib | | | | | |
| | | | | | or Cmpd I | | | | Combination | |
| | | | | | (Lapatinib + | | | | Effect | |
| | | HER | Erlotinib | | Cmpd I) | | Cmpd I | | CI @IC50 | |
| Cell lines | cMET | amp+ | −HGF | +HGF | −HGF | +HGF | −HGF | +HGF | −HGF | +HGF |
| breast_KPL4 | cMET-low | HER2+ | >10 | >10 | 0.21 | 0.19 | 0.47 | 0.59 | NA | NA |
| colon_HT29 | cMET-over | no | >10 | >10 | 0.43 | 0.47 | 0.56 | 0.57 | NA | NA |
| colon_SW48 | cMET-over | no | 2.35 | 2.62 | 0.12 | 0.09 | 0.23 | 0.18 | 0.56 | 0.44 |
| gastric_AGS | cMET-low | no | >10 | >10 | 0.30 | 0.31 | 0.61 | 0.61 | NA | NA |
| gastric_SNU16 | cMET-over | no | 6.46 | 9.37 | 0.05 | 0.06 | 0.06 | 0.07 | 0.86 | 0.78 |
| hn_Detroit562 | cMET+ (<5) | no | >10 | >10 | 0.25 | 0.16 | 0.41 | 0.37 | NA | NA |
| hn_HN5* | cMET-over | HER1+ | 6.72 | >10 | 0.13 | 0.21 | 0.80 | 1.09 | 0.18 | NA |
| hn_HN5C2* | cMET-over | HER1+ | >10 | >10 | 0.21 | 0.29 | 0.81 | 1.33 | NA | NA |
| hn_SCC12 | cMET-over | no | >10 | >10 | 0.37 | 0.44 | 1.14 | 1.36 | NA | NA |
| hn_SCC15 | cMET-over | HER1+ | 1.62 | 7.70 | 0.13 | 0.14 | 0.63 | 0.61 | 0.30 | 0.25 |
| lung_H1573 | cMET+ | HER1+ | >10 | >10 | 0.43 | 0.34 | 1.51 | 1.03 | NA | NA |
| lung_H1648 | cMET+ | no | >10 | >10 | 0.33 | 0.06 | 0.96 | 0.40 | NA | NA |
| lung_H1975 | TBD | no | >10 | >10 | 0.98 | 0.99 | 1.39 | 1.22 | NA | NA |
| lung_H1993* | cMET+ | no | 8.13 | >10 | 0.004 | 0.02 | 0.01 | 0.04 | 0.32 | NA |
| lung_H2170 | cMET-over | HER2+ | 1.28 | 7.74 | 0.33 | 0.30 | 0.67 | 0.53 | 0.90 | 0.61 |
| lung_H2342 | cMET+ | no | >10 | >10 | 0.84 | 0.79 | 1.61 | 1.62 | NA | NA |
| lung_H441 | cMET+ (<5) | no | >10 | >10 | 0.61 | 0.62 | 1.26 | 1.44 | NA | NA |
| lung_H596 | cMET-mut | no | >10 | >10 | 0.59 | 0.44 | 1.22 | 0.82 | NA | NA |
| lung_H69 | cMET-mut | no | >10 | >10 | 0.90 | 0.79 | 1.20 | 1.05 | NA | NA |
| lung_HOP-92 | cMET-mut | no | >10 | >10 | 0.45 | 0.35 | 0.82 | 0.65 | NA | NA |
| ovary_SKOV3 | cMET-over | HER2+ | 5.62 | 5.39 | 0.84 | 0.78 | 1.64 | 1.54 | 0.74 | 0.72 |
| skin_A431* | cMET-over | HER1+ | 3.22 | >10 | 0.18 | 0.20 | 0.50 | 0.52 | 0.44 | NA |

*N = 1, one experiment was performed.

The Combination Effects of Compound I with Lapatinib or Anti-HER3 Antibody on HER3 Over-Expressed Tumor Cell Lines.

MKN45 cells have cMET+ and overexpressed level of HER3. As shown in Table 7 and FIG. 9, HRG reduced the sensitivity of Compound I to inhibit cell growth (the IC₅₀ value increased from 20 nM in the absence of HRG to 450 nM in the presence of HRG) and phosphorylation of HER3 in MKN45 tumor cells. Unexpectedly, lapatinib restored Compound I sensitivity and showed strong synergy of cell growth inhibition as indicated as CI=0.12 and EOHSA analysis when it combined with Compound I in the presence of HRG in MKN45 cells. As a control, HS746T gastric tumor cells with MET+ and low expression of HER3 remained sensitive to Compound I even in the presence of HRG. The above results demonstrate that combining Compound I with lapatinib is beneficial in MET+ and HER3-over-expressed tumor cells. Further, combining Compound I with an anti-HER3 antibody (monoclonal antihuman erbB3 antibody mab3481, available from R&D Systems, Minneapolis, Minn.) increased the sensitivity of Compound I and showed a synergistic effect (EOHSA) on cell growth inhibition in MKN45 cells (Table 8).

TABLE 7
Cell growth inhibition effect of Compound I in combination with lapatinib in
MET+ and HER3 over-expressed tumor cell lines.

| | | Average IC50 (μM) N = 2 | | | | | | | |
|---|---|---|---|---|---|---|---|---|---|
| | | | | Lapatinib | | | | | |
| | | | | or Cmpd I | | | | Combination | |
| | | | | (Lapatinib + | | | | Effect | |
| | | Lapatinib | | Cmpd I) | | Cmpd I | | CI @IC50 | |
| Cell lines | HER3 | −HRG | +HRG | −HRG | +HRG | −HRG | +HRG | −HRG | +HRG |
| MKN-45 | HER3- | 6.16 | 5.06 | 0.01 | 0.04 | 0.02 | 0.45 | 0.75 | 0.12 |
| | over | | | | | | | | |
| HS746T | HER3- | 7.69 | 7.37 | 0.01 | 0.01 | 0.01 | 0.01 | 0.86 | 0.73 |
| | low | | | | | | | | |

TABLE 8
Cell growth inhibition effect of Compound I in combination with
anti-HER3 antibody in HER3 over-expressed MKN-45 tumor cell line.

| | | | +HRG (10 ng/mL), Average IC₅₀) N = 2 | | |
|---|---|---|---|---|---|
| | | | anti-HER3ab | anti-HER3ab (μg/mL) or Cmpd I | Cmpd I |
| Cell lines | cMET | HER3 | (μg/mLl) | (μM) (anti-HER3ab + Cmpd I) | (μM) |
| MKN-45 | CMET+ | HER3-over | >10 | 0.05 | 0.47 |

### Effect of Compound I and Gefitinib on Tumor Cell Lines.

Gefitinib is a selective HER1 inhibitor. Gefitinib alone was not very active in the two lung tumor cell lines tested and showed moderate activity in the SCC15 head and neck tumor line. Combination of Compound I and gefitinib showed synergy of cell growth inhibition as indicated as CI<0.9 and/or EOHSA analysis in the lung and head and neck tumor cell lines listed in Table 9.

TABLE 9
Cell growth inhibition effect of Compound I and Gefitinib combination at 1:1
constant molar ratio on lung and head and neck tumor cell lines.

| | | | Average IC50 (μM) N = 2 | | | | | | | |
|---|---|---|---|---|---|---|---|---|---|---|
| | | | | | Gefitinib | | | | | |
| | | | | | or Cmpd I | | | | Combination | |
| | | | | | (Lapatinib + | | | | Effect | |
| | | HER | Gefitinib | | Cmpd I) | | Cmpd I | | CI @IC50 | |
| Cell lines | cMET | amp+ | −HGF | +HGF | −HGF | +HGF | −HGF | +HGF | −HGF | +HGF |
| lung_H1648 | cMET+ | no | 10.28 | >10 | 0.18 | 0.12 | 0.85 | 0.62 | 0.15 | NA |
| lung_H1573 | cMET+ | HER1+ | >10 | >10 | 0.52 | 0.37 | 1.75 | 1.12 | NA | NA |
| hn_SCC15 | cMET-over | HER1+ | 1.21 | 5.44 | 0.10 | 0.12 | 0.67 | 0.82 | 0.25 | 0.17 |

What is claimed is:

1. A method of treating breast cancer in a patient comprising administering to the patient therapeutically effective amounts of:
a) a compound of the Formula I: 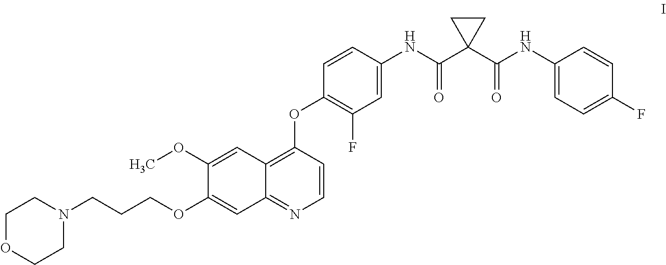 [SMILES: COc1cc2c(Oc3ccc(NC(=O)C4(C(=O)Nc5ccc(F)cc5)CC4)cc3F)ccnc2cc1OCCCN1CCOCC1]
or a pharmaceutically acceptable salt thereof and an erbB-2 inhibitor.

2. The method of claim 1 wherein a tumor cell from said breast cancer expresses cMET.

3. The method of claim 1, wherein a tumor cell from said breast cancer overexpresses erbB-2.

4. The method of claim 1, wherein said erbB-2 inhibitor comprises a compound of Formula II 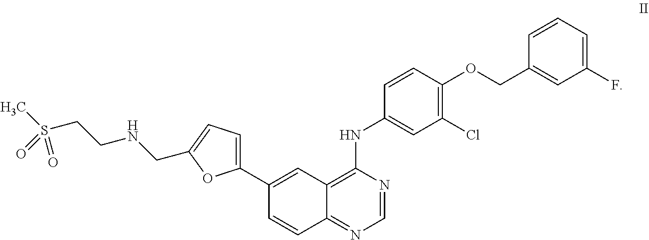 [SMILES: CS(=O)(=O)CCNCc1ccc(-c2ccc3ncnc(Nc4ccc(OCc5cccc(F)c5)c(Cl)c4)c3c2)o1]
or a pharmaceutically acceptable salt thereof.

5. The method of claim 1, wherein said erbB-2 inhibitor comprises trastuzumab.

6. The method of claim 1, wherein said breast cancer is resistant to lapatinib or a pharmaceutically acceptable salt thereof or trastuzumab.

7. The method of claim 1, wherein said compound of Formula I and said erbB-2 inhibitor have a synergistic effect in inhibiting cell growth said breast cancer.

8. The method of claim 1 wherein a tumor cell from said breast cancer expresses AXL.

9. The method of claim 8, wherein the tumor cell overexpresses AXL.

10. A method of treating cancer in a patient in need thereof, comprising identifying if a tumor cell from said cancer expresses or overexpresses AXL and if said tumor cell expresses or overexpresses AXL administering a composition comprising Formula I and an erbB-2 inhibitor to said patient.

11. The method of claim 10 wherein said cancer is resistant to lapatinib or a pharmaceutically acceptable salt thereof.

12. The method of claim 10 wherein said cancer is resistant to trastuzumab.

13. The method of claim 10 wherein said erbB-2 inhibitor is lapatinib or a pharmaceutically acceptable salt thereof.

14. The method of claim 10 wherein said erbB-2 inhibitor is trastuzumab.

15. The method of claim 10 wherein said cancer is breast cancer.
